(19) United States
(12) Patent Application Publication
(10) Pub. No.: US 2007/0284182 A1
(43) Pub. Date: Dec. 13, 2007

(54) Waterproof earplug with sound-transmitting effect

(76) Inventor: Chia-Chun Mu, Taipei Hsin (TW)

Correspondence Address: TROXELL LAW OFFICE PLLC;Suite 1404, 5205 Leesburg Pike, Falls Church, VA, 22041, US

(21) Appl. No.: 11/450,467

(22) Filed: Jun. 12, 2006

(51) Int. Cl. H04R 25/02; A61B 7/02

(52) U.S. Cl. 181/135; 181/130

(57) ABSTRACT

A waterproof earplug with sound-transmitting effect comprises mainly a pull stub and a sound-barrier membrane; the sound-barrier membrane is substantially a thin round membrane; the pull stub is furnished in the center of the sound-barrier membrane, and it extends outwards at a given length so as to facilitate a hand to grip; the sound-barrier membrane is formed into a round membrane so as to fit the curved surface of the inner helix closely to provide a waterproof effect; after the round membrane of the earplug is in close contact with the inner helix, a sound from the outside of the ear can still be tansmitted therein as a result of the resonant effect of the round membrane.

### BACKGROUND OF THE INVENTION

1. Field of the Invention

This invention relates to an earplug, and particularly to a waterproof earplug with sound-transmitting effect.

2. Description of the Prior Art

A conventional earplug usually comprises a nest of round membranes on a pull stub, and the size of each round membrane is increased from the inside to the outer one so as to fit size of all person's inner helix; every two round membranes form into a space of sound barrier; as soon as an earplug is plugged into the inner helix, the outside sound can not go through the space of sound barrier.

A conventional earplug comprises a pull stub, of which the outer surface is furnished with multi-round strata; the space between every two stratam is very short; after the earplug is plugged into the inner helix, the multi-round strata will be in close contact with the inner helix so as to provide a waterproof effect; however, the space among the round strata form into a sound-barrier layer to blockade outside sound from transmitting into the ear.

### SUMMARY OF THE INVENTION

The prime object of the present invention is to provide a waterproof earplug, in which the front end of the pull stub has a sound-barrier membrane, and the sound-barrier membrane is substantially a round membrane; after the earplug is plugged into the inner helix, the round membrane will be in close contact with the inner helix completely so as to provide a waterproof effect; since the sound-barrier membrane is a thin and round membrane, outside sound can be transmitted into the ear by means of the resonant effect of the thin and round membrane.

Another object of the present invention is to provide a waterproof earplug, in which the sound-barrier membrane is a single layer of round membrance; the pull stub in the thin and round membrane is molded into one piece with the round membrane; the sound-barrier membrane includes two different round membranes in terms of size, and they are partitioned with a partition line; as soon as the earplug is plugged into an ear, the inner round membrane will first be in contact with the inner helix closely so as to provide a waterproof effect as desired.

Still another object of the present invention is to provide a waterproof earplug, in which the sound-barrier membrane includes two different round membranes in terms of size; a separation line is furnished between the two round membranes, and the size of the separation line is designed to fit the largest size of an inner helix; when the earplug is plugged into an inner helix, the surface of the round membranes on both sides of the separation line will be in close contact with the inner helix without feeling any pressure and tension.

A further object of the present invention is to provide a waterproof earplug, in which the center of the sound-barrier membrance has a pull stub extended out of the sound-barrier membrane; one end of the pull stub is furnished with a flange ring and a cylindrical hole; the flange ring facilitate to grip the earplug, and cylindrical hole is used for connecting a string.

### BRIEF DESCRITPION OF THE DRAWINGS

FIG. 1 is a perspective view of the present invention.

FIG. 2 is a sectional view of the present invention.

FIG. 3. is a sectional view-1 of the present invention, showing the earplug being plugged into ear.

FIG. 4 is a sectional view-2 of the present invention, showing the earplug being plugged into ear.

FIG. 5 is a sectional view-3 of the present invention, showing the earplug being further plugged into ear.

FIG. 6 is an enlarged sectional view of the present invention, showing the earplug being in close contact within the ear.

### DETAILED DESCRIPTION OF THE PREFERRED EMBODIMENTS

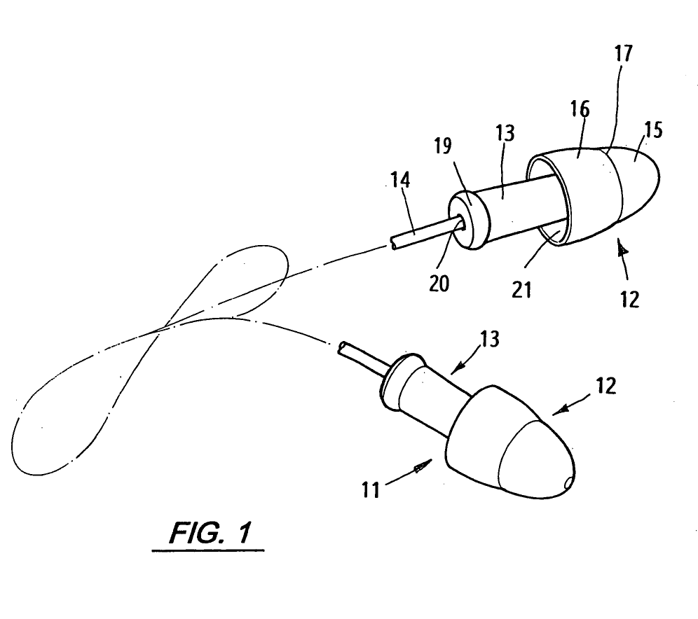

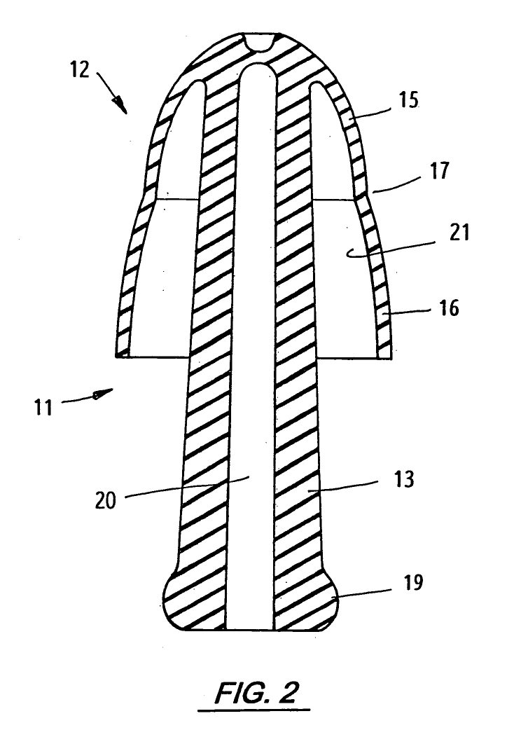

This invention relates to a waterproof earplug with sound-transmitting effect; as shown in FIGS. 1 and 2, the earplug 11 comprises two plugs as one set; each earplug includes a sound-barrier membrane 12, a pull stub 13 having a cylindrical hole 20 in the center thereof, The cylindrical hole 20 is used for receiving a string 14 so as to have the two earplugs connected together. Each earplug includes a sound-barrier membrane 12 and a pull stub 13, which are molded into one piece; the sound-barrier membrane 12 is substantially a round membrane; the pull stub 13 is furnsihed in the center of the sound-barrier membrane 12, and extended outward at a given length so as to facilitate gripping with hand. Since the sound-barrier membrane 12 is a round membrane, it can be in close contact with the curve in the inner helix of an ear so as to provide the earplug 11 with a waterproof function. After the round membrane is mounted in the helix, a person still can hear a sound out of the ear because of the round membrane having a resonant effect.

Referring to drawing 1 to 6, the sound-barrier membrane 12 and the pull stub 13 of the earplug 11 are molded into form by means of a silicone; the pull stub 13 in the center of the sound-barrier membrane 12 is an elongate bar with a given length; one end of the pull stub is connected together with the sound-barrier membrane 12, while the other end thereof is furnished with a flange ring 19; the center of the pull stub 13 has a cylindrical hole 20; the pull stub is designed to set in the center of the sound-barrier memnbrane 12 so as to facilitate gripping with hand to plug the earplug 11 in an ear.

The sound-barrier membrane 12 at one end of the pull stub 13 is a single layer of round membrane, which includes at least two different round membranes 15 and 16, and there is a separation line 17 between them so as to fit the size of the inner helixes of the most people; after the earplug 11 is plugged in one's inner helix, the round membranes 15 and 16 will fit in the inner helix around the separation line 17 without having any pressure to the ear.

The inner round membrane 15 at the front end of the sound-barrier membrane 12 is a small round membrane, and the front end thereof is formed into a round and curved shape. After the earplug 11 is plugged into the ear, the inner round membrane 15 will fit to the shape of the inner helix closely, i.e., the round membrane would be in close contact with the inner surface of the ear.

The outer round membrane 16 is furnished at the rear end of the sound-barrier membrane 12, and it is separated from the inner membrane 15 by means of a separation line 17 so as to provide the sound-barrier membrane 12 with two different round membranes. After the earplug 11 is plugged into an ear, the inner round membrane 15 would go into the ear to fit to the ear shape; if the inner round membrane 15 is unable to fit the inner helix closely, the outer round membrane 16 will fit the inner helix closely and completely so as to have the sound-barrier membrane 12 fitted the ear of every person.

The sound-barrier membrane 12 of the earplug 11 is a round membrane with a pull stub in the center thereof; the pull stub 13 has a suitable diameter; an inner membrane space 21 is furnished between the sound-barrier membrane 12 and the pull stub 13. After the earplug 11 is plugged into the inner helix, there is only a given part thereof is in close contact with the inner helix; in that case, the outer end of the sound-barrier membrane 12 is left outside the ear without kindering sound to be transmitted into the ear. When a sound enters the inner membrane space 21, it would have a resonant effect to have a sound transmitted into the ear; simultaneously, since the sound-barrier membrane 12 and the inner helix are in close and hermetic contact, no water can enter the ear, i.e., a person with an earplug 11 can still hear the outside sound.

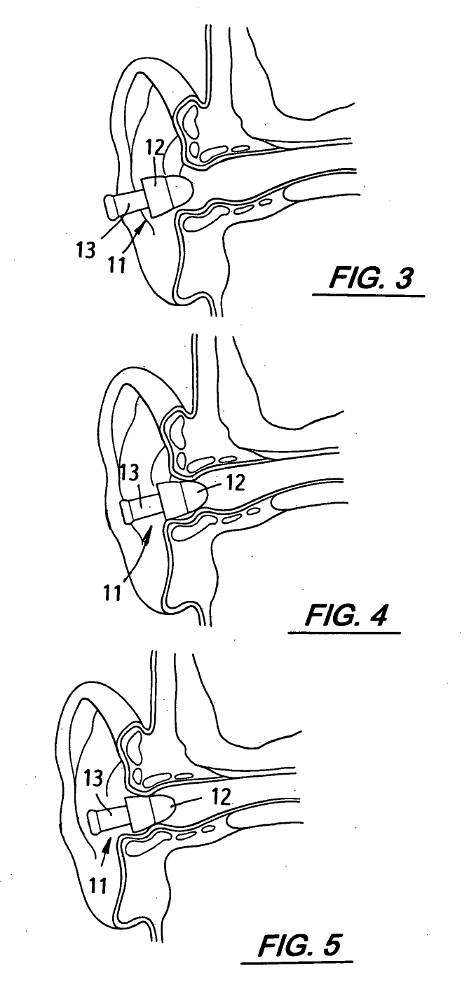

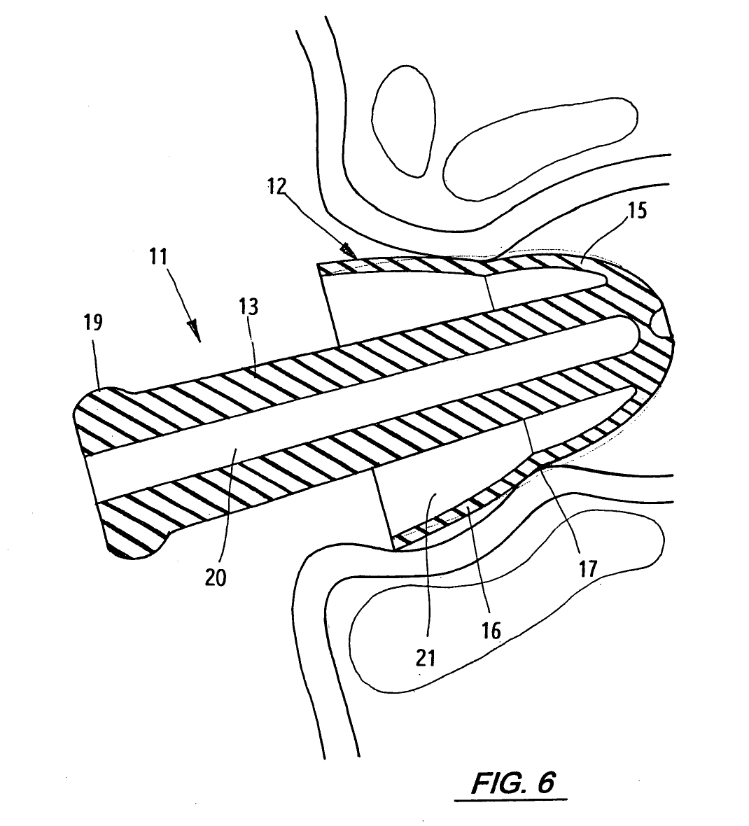

Referring to FIGS. 3 to 5, the outer part of the inner round membrane 15 of the sound-barrier membrane 12 would be in contact with the inner helix first upon an earplug being plugged in the ear. After the inner round membrane 15 is in contact with the ear, the earplug 11 will enter the ear comfortably without feeling any pressure; then, the sound-barrier membrane 12 would be in close contact with the ear to provide a waterproof function. As shown in FIG. 6, the earplug 11 is set in a close contact condition comfortably without feeling any pressure by means of a single layer of membrane, but the outside sound can enter the ear as a result of resonant effect in the inner membrane space 21 of the sound-barrier membrane 12 without hindering the waterproof function; in other words, two persons can talk and hear each other upon the earplugs being mounted in their ears.

According to the aforesaid detailed description of the preferred embodiment, the features and structure relation of the present invention have been disclosed completely, and it is apparent that the present invention has an evident improvement of the kind, and it is never anticipated and accomplished before by anyone who is skilled in the field; therefore,the structure of the present invention is deemed unique.

While the invention has been described with reference to specific embodiments it must be understood that those embodiments are susceptible to many changes, substitutions, and modifications that will be readily apparent to those having ordinary skill in the art without departing from the scope and spirit of the invention.

What is claimed is:

1. A waterproof earplug with sound-transmitting effect, comprising:
a pull stub having a flange ring at one end thereof, and other end thereof molded with center of a sound-barrier membrane into one piece;
a sound-barrier membrane being substantially a single layer of membrane furnished at one end thereof, and said sound-barrier membrane including at least two different round membranes;

2. A waterproof earplug with sound-transmitting effect as claimed in claim 1, wherein said two different round membranes is partitioned with a separation line.

3. A waterproof earplug with sound-transmitting effect as claimed in claim 1, wherein said two different round membranes include a small one and a large one arranged in a given order.

4. A waterproof earplug with sound-tansmitting effect as claimed in claim 1, wherein center of said sound-barrier membrane is furnished with a pull stub and an inner membrane space.
